(19) United States
(12) Patent Application Publication
(10) Pub. No.: US 2010/0081958 A1
(43) Pub. Date: Apr. 1, 2010

(54) PULSE-BASED FEATURE EXTRACTION FOR NEURAL RECORDINGS

(76) Inventor: Christy L. She, Allen, TX (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/444,008

(22) PCT Filed: Oct. 2, 2007
(86) PCT No.: PCT/US07/80190

§ 371 Date: Nov. 4, 2009

Related U.S. Application Data
(60) Provisional application No. 60/848,671, filed on Oct. 2, 2006.

(51) Int. Cl. A61B 5/0476; G06G 7/18

(52) U.S. Cl. 600/544; 327/336; 327/344

(57) ABSTRACT

A neural recording system (100) and method (400) for neural encoding is provided. The system can include an ultra-low power neural encoder (120) for compressing spikes within a neural signal (110) to produce a pulse train (130) and wirelessly transmitting the pulse train to a spike sorter (140). Features of the neural signal can be encoded such that the timing between pulses and the number of pulses conveys features of the spike. The neural encoder can include an Integrate and Fire (IF) neuron 230 that performs spike detection and encodes at least one spike (112) of the neural signal. A leakiness aspect (232) and an adaptive aspect (337) can be included with the IF circuit for combining aspects of spike detection and spike sorting for suppressing noise, keeping power consumption low, and improving signal resolution.

### FIELD OF THE INVENTION

The present invention relates to the field of signal processing, and more particularly, to recording and processing neural signals.

### BACKGROUND

Neurophysiology studies are directed towards understanding the nervous system. Such studies can include identifying the mechanisms of neural activity in the brain. Neural data acquisition systems can assist neurophysiologists in identifying neural activity to help diagnosis and treat patients. As one example of a data acquisition system, electrodes can be placed on, or inserted into, nerve tissue for recording neural activity. Neurophysiologists can analyze the recorded neural signals to recognize differing brain activities. The brain activity is the result of many neurons communicating with one another. Neurons are cells within the brain responsible for transmitting and receiving electrical signals. The electrical signals can be conveyed throughout the nervous system to provide motor movement function or other central nervous system activities.

Many neuroscience applications are devoted to the analysis of spike trains, which reflect the firing of neurons. The firing of a neuron occurs when a neuron generates an action potential in response to an electrical stimuli. The electrical stimuli is associated with activity generated from the neuron or from activity generated by a group of neurons. The action potential is considered a spike and can be visualized as a voltage signal from an electrode recording. A single electrode can record spikes from more than one neuron; however, this can increase the difficulty of discriminating between spike features since features from multiple neurons are captured together. A spike is broadly defined as a sharp transient that is visibly different from the background noise activity. Although, neuroscientists debate how neural spikes represent neural information, it is believed that each spike includes features characteristic to a neuron originating the neural spike.

A brain-machine interface (BMI) is a type of neural data acquisition system that can extract information from neural recordings of the brain. As an example, the BMI can capture neural activity in the motor cortex with the goal of creating predictive models for hand movement and directly controlling a robotic device. Current instrumentation technology and surgical procedures for BMI allow for neural recordings from hundreds of electrodes at once. For example, a neurosurgeon can place a gird of electrodes on cortical tissue to record neural activity. The electrodes are usually connectively wired to a computer for recording the neural activity. The recordings from each electrode however can require a significant amount of memory to store (one channel is typically sampled at 25 kHz, 16 bits). Transferring the large bandwidth data streams associated with the neural recordings can also require the subject to be tethered to numerous wires. Recording neural signals from the patient is thus a patient centric procedure.

Moreover, it is not certain as to which features of recorded neural signals within the large bandwidth data streams are relevant to neural activity. Neural signal data reduction is a classical problem in neuroscience that is concerned with compressing the amount of data needed to represent the neural signal prior to transmitting the data for analysis. One prior art method is to wirelessly transmit a segment of the raw waveform surrounding the spike, and then sort the spikes outside the subject where power and size constraints are less stringent. The segment occupies less memory than the entire waveform. However, this requires significant memory and processing power as portions of the raw waveform are still transmitted. Another prior art method is to extract and send various features of the waveform themselves for analysis outside the subject. The spike can be represented by a parametric model whereby the parameters of the model are transmitted. The parametric model can however consume significant processing power which is limited on a medical device. Yet another method for low-bandwidth communication involves transmitting only spike times or binned spike counts. However, this method does not allow for spike sorting. Effective solutions to any of these methods can require significant memory capacity and power consumption. Each of the proposed data reduction techniques known in the art either dissipates too much power for an implanted device and/or does not allow for spike sorting. Accordingly a need for a low-power low-bandwidth device for neural signal data acquisition and analysis is needed.

### SUMMARY

Broadly stated, embodiments of the invention are directed to a neural acquisition system, a neural encoder, and a method for efficiently encoding and wirelessly transmitting encoded neural signals for spike detection. The neural acquisition system can include the neural encoder for temporal-based pulse coding of a neural signal, and a spike sorter for sorting spikes encoded in the temporal-based pulse coding. The neural encoder can generate a temporal-based pulse coded representation of spikes in the neural signal based on integrate-and-fire coding of the received neural signal. The neural encoder can include spike detection and encode features of the spikes as a timing between pulses such that the timing between pulses represents features of the spikes. The spike sorter can receive the temporal-based pulse coded representation and identify neurons generating the spikes from the temporal-based pulse coded representation. The spike sorter can identify neurons directly from the temporal-based pulse coded representation without reconstructing the neural signal.

The neural encoder can include a processor for generating the temporal-based pulse coded representation of spikes from the neural signal, a transmitter operatively coupled to the processor for wirelessly communicating the temporal-based pulse coded representation, and a power source for ultra-low powering of the processor and the wireless module. The spike sorter can include a receiver for wirelessly receiving the temporal-based pulse coding from the neural encoder. The neural encoder and the spike sorter can operate asynchronously to increase a resolution of the neural signal. In one arrangement, the spike sorter can operate directly on the timing of pulses for sorting spikes to avoid reconstruction of the neural signal. The spike sorter can include a cluster based classifier for synchronizing spike signatures, comparing the spike signatures to templates associated with neurons, and identifying a neuron producing a spike signature. The spike sorter can classify a spike signature and identify a neuron. The neural encoder can include a bank of Integrate and Fire (IF) neurons tuned to different frequency bands to span a range for temporal-based pulse coding of the neural signal.

Embodiments of the invention also include a neural encoder. The neural encoder can include an electrode for capturing a neural signal and at least one Integrate and Fire (IF) circuit. The IF circuit can model at least one spike of the neural signal and generate a pulse train in accordance with a waveform of the spike. The IF circuit can introduce a timing between pulses of the pulse train for encoding at least one feature of the waveform. In one aspect, the IF circuit can model an area, size, or shape of the waveform as a feature to establish the timing between pulses of the pulse train. For example, the IF circuit can decrease a period of the pulses for wide spikes, and increase a period of the pulses for narrow spikes. The IF circuit can decrease a period of the pulses for high-amplitude spikes, and increase a period of the pulses for low-amplitude spikes. In one arrangement, the IF circuit can also be configured as a leaky integrator (LIF) circuit. The LIF circuit includes leaky integration for synchronizing spike signatures and increasing a robustness to noise. The LIF circuit can include at least one user setting for adjusting a bandwidth compression of the bi-phasic output pulse train. An adaptive aspect can also be introduced to the LIF circuit for adjusting a timing and number of pulses for bandwidth compression.

Embodiments of the invention also include a Leaky and Integrate Fire (LIF) circuit. The LIF circuit can include a leaky integrator for providing a leakiness to an integration of a neural signal, and a pulse generator for producing a pulse train of the neural signal from the leaky integration. The leaky integrator can include a capacitor for building up a charge in accordance with a voltage of the neural signal, and a resistor coupled in parallel with the capacitor that leaks off a portion of the charge. The resistor provides a leakiness to the integrating by decreasing the charge on the capacitor over time. In one arrangement, the pulse train can be bi-phasic. Features of the spike can be encoded as a timing between pulses of the bi-phasic output pulse train such that the timing between pulses conveys features of the spike. In one arrangement, the pulse generator can include a bi-phasic comparator for generating a positive pulse output and a negative pulse output when the leaky integration exceeds at least one threshold, and an OR gate coupled to the positive pulse output and negative pulse output for resetting the circuit after a pulse. The capacitor and the resistor, when arranged in parallel, provide input to the bi-phasic comparator such that an input to the LIF circuit produces the bi-phasic output pulse train.

The bi-phasic comparator can include a first comparator for generating a positive pulse output, and a second comparator for generating a negative pulse output. The first comparator can include a first adjustable threshold for setting a pulse rate based on a positive portion of the signal's area of a spike. Similarly, the second comparator can include a second adjustable threshold for setting a pulse rate based on a negative portion of the signal's area of a spike. The LIF circuit can include a feedback unit coupling the output of the amplifier to the input of the amplifier for adjusting a timing between pulses of the bi-phasic output pulse train. The feedback unit can include a delay element to increase a timing between pulses of the bi-phasic output pulse train for modeling a neural refractory period. The feedback unit can also include an adaptive unit for monitoring a pulse rate and adjusting a threshold of the amplifier to limit the pulse rate. For example, the adaptive unit can increase the threshold for increasing pulse rates to lessen a number of generated pulses, and decrease the threshold for decreasing pulse rates to increase a number of generated pulses.

Other embodiments of the invention also include a method for neural encoding. The method can include the steps of integrating a neural signal, comparing the integration to a threshold, and generating a pulse if the integration exceeds the threshold. In one aspect, a leakiness can be introduced to the integrating to suppress noise on the spike. The method can further include wirelessly transmitting the pulse train asynchronously to a spike sorter. In such regard, the pulse train provides bandwidth compression of the neural signal. The method can further include enabling a power amplifier to transmit a pulse when the leaky integration exceeds a threshold, keeping the power amplifier in power save mode so as to otherwise provide ultra-low power consumption. The method can further include the sorting of spikes encoded within the timing of the pulse train without reconstructing the neural signal. The comparing can include comparing the leaky integration to a positive threshold and generating a positive pulse if the leaky integration exceeds the positive threshold, and comparing the leaky integration to a negative threshold and generating a negative pulse if the leaky integration exceeds the negative threshold. The generating of a pulse train can include adjusting a pulse rate in accordance with an area of a waveform of the spike, or adjusting a pulse rate in accordance with an amplitude of a waveform of the spike. The generating of a pulse train can include introducing a delay in a feedback of the pulse train for modeling a refractory period, or adapting the threshold in accordance with the timing between pulses for modeling inhibition and excitation.

### BRIEF DESCRIPTION OF THE DRAWINGS

Various features of the system are set forth with particularity in the appended claims. The embodiments herein, can be understood by reference to the following description, taken in conjunction with the accompanying drawings, in which:

FIG. 1 is a schematic diagram of a neural recording system in accordance with one embodiment of the invention;

FIG. 2 is a plot of a neural signal showing multiple spikes in accordance with one embodiment of the invention;

FIG. 3 is a plot of a pulse train in accordance with one embodiment of the invention;

FIG. 4 is a block diagram of a neural encoder in accordance with the invention;

FIG. 5 is a block diagram of a spike sorter in accordance with the invention;

FIG. 6 is a block diagram of the processor of the neural encoder of FIG. 5 in accordance with the invention;

FIG. 7 is schematic of a leaky integrate-and-fire (LIF) circuit in accordance with the invention;

FIG. 8 is a circuit of the LIF circuit of FIG. 8 in accordance with the invention;

FIG. 9 is a method for neural encoding in accordance with the invention;

FIG. 10 is a plot of a neural signal showing multiple spikes in accordance with the invention;

FIG. 11 is a plot of the pulse trains produced from encoding the multiple spikes of the neural signal of FIG. 11 in accordance with the invention;

FIG. 12 is zoomed in view of a pulse train for a single spike in accordance with the invention;

FIG. 13 is a zoomed in view of another pulse train for a single spike in accordance with the invention;

FIG. 14 is an overlay plot of three spike signals having varying amplitude and area in accordance with the invention;

FIG. 15 is a noisy version of the neural spike; and

FIG. 16 is an illustration for each of the three pulse trains produced from the encoding of the spike signals of FIG. 15 and each of the three pulse trains produced from the encoding of the corresponding noisy spike signals of FIG. 16 in accordance with the invention.

### DETAILED DESCRIPTION

Embodiments of the invention are directed to a pulse-based neural recording system. The pulse-based neural recording system can provide advantages in terms of low power and low bandwidth. In a first aspect, spike detection can be performed by a neural encoder that generates electronic pulses for detected neural spikes in a neural signal. In a second aspect, the neural encoder can perform Integrate-and-Fire coding to convey a sufficient number of pulses per unit time to permit accurate reconstruction of the neural signal. The pulses can then be wirelessly transmitted to a spike sorter that analyzes the pulses. This offers a low transmission bandwidth since spike sorting does not need to be performed at the sensor end. Moreover, the pulse-based neural recording system sends just enough pulses as needed to allow for spike sorting at the spike sorter but much less than are needed for a complete reconstruction of the neural signal thereby providing efficient bandwidth compression.

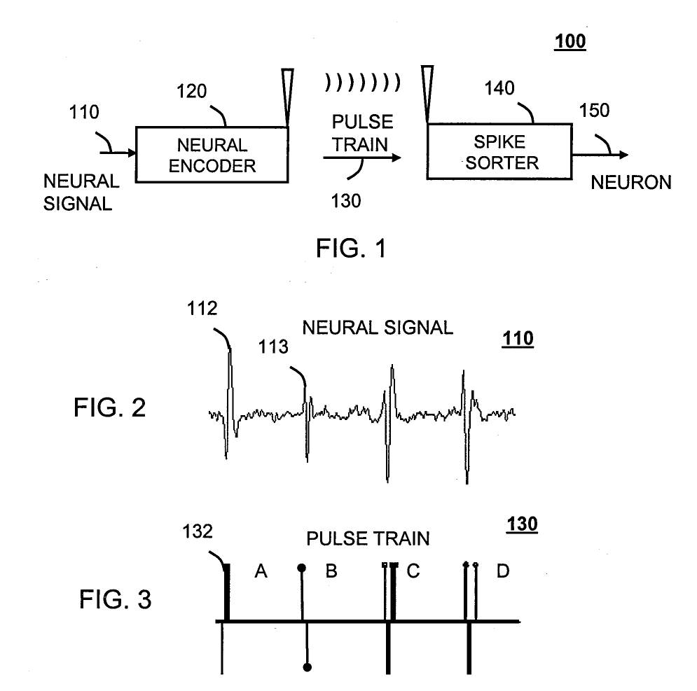

Referring to FIG. 1, a pulse-based neural recording system 100 is shown. The neural recording system 100 can provide an ultra-low power operation for extracting spike information from neural signals 110 and transmitting the spike information at a reduced bandwidth. Two modules of the neural recording system are provided although other modules are contemplated: a neural encoder 120 for temporal based pulse coding of spikes in the neural signals 110, and a spike sorter 140 for classifying the spikes encoded in the temporal based pulse coding. As shown, the neural recording system 100 can acquire the neural signals 110, generate a pulse train 130 representing the neural signals 110, wirelessly transmit the pulse train 130, detect and sort spikes from an analysis of the pulse train 130, and generate an output 150 that identifies spikes or characterizes spike information.

In the following, the terms and phrases used herein are not intended to be limiting but rather to provide an understandable description of the embodiment herein. The term “neural signal” can be defined as a waveform captured from an electrode in neurophysiology recordings. The term “spike” can be defined as a high-amplitude time varying waveform in a neural signal. The term “pulse” can be defined as a component used for coding one of more features of a spike in a neural signal. The term “pulse train” can be defined as a sequence of pulses in time. The term “feature” can be defined as an attribute of a spike, for example, an amplitude, width, area, or shape of a spike. The pulse train provides a bandwidth compression of the neural signal and is suitable for use in ultra-low power consumption devices. The term “feature” can be defined as an attribute of a neural signal, for example, an amplitude, width, area, or shape.

The neural encoder 120 can encode neural spike information into the pulse train 130 by representing the neural spikes as a timing between pulses and a number of pulses. The neural encoder 120 can be an implantable device that attaches to a portion of biological tissue, or an external device electrochemically coupled to a portion of biological tissue, such as brain tissue or nerve tissue. As one example, the neural encoder 120 can be a neural micro-device implanted within the cortex of a human subject. An electrode operatively coupled to the neural encoder 120 can capture the neural signal 110. The neural encoder 120 can wirelessly transmit the pulse train 130 to the spike sorter 140. As such, the neural encoder 120 can provide ultra-low power and robust analog spike feature extraction by encoding the neural signals 110 as the pulse train 130. The encoding can significantly reduce the neural signal's bandwidth prior to transmission to the spike sorter 140.

The spike sorter 140 can analyze the timing information and number of pulses in the received pulse train 130 to sort the encoded spikes. As one particular advantage, the spike sorter 140 can operate directly on the pulse train 130 without regenerating the neural signal 110. This allows the spike sorter 140 to categorize spikes encoded by time and position in the pulse train 130, and produce an output 150 that identifies at least one spike in the neural signal. The spike sorter 140 can also generate an output 150 that identifies a type or location of a neuron generating the one or more spikes. The term “spike detection” can be defined as identifying the presence of a spike in a neural signal. The term “spike sorter” can be defined as categorizing pulses in a coded signal for associating the pulses with a particular spike in a neural signal.

Referring additionally to FIG. 2, an exemplary neural signal 110 is shown. The neural signal 110 can be captured from an electrode or any other suitable electrophysiological monitoring or recording equipment. The neural signal 110 can include one or more spikes 112 and 113, such as an action potential, associated with neural activity. Moreover, each spike and the attributes of each spike (e.g. width, height, area, etc.) can be associated with a particular neuron. For example, a first neuron may be responsible for generating spike 112, and another neuron may be responsible for generating spike 113. The neural encoder 120 can detect spikes 112 and 113 within the neural signal 110 prior to generating the pulse train 130 (e.g. compression) to avoid coding of noise or periods of neural non-activity. The neural encoder 120 can encode the neural signal 110 and produce the pulse train signal 130.

Referring to FIG. 3, an exemplary pulse train 130 is shown. In particular, each spike (e.g. 112 and 113) within the neural signal 110 can be represented each as a group 132 of pulses in the pulse train 130. The neural encoder 120 can generate the pulse train 130 from the neural signal 110. The timing between the pulses and the number of pulses in the group 132 of pulses convey features of the spike 112. For instance, the timing and number of pulses can be associated with the amplitude, area, width, or shape of the spike 112 but is not limited to thereof. The timing of the pulses in each pulse group 132 can thus be used to identify particular neurons (e.g., number, position) or types of neuron (e.g., cell structure, size). In particular, features of the neural signal 110 are encoded in the timing between pulses and the number of pulses in the pulse train signal 130. By way of the neural encoder 120, spatial information related to features of the neural signal 110 can be transformed to temporally-encoded information in the pulse train signal 130. The temporal encoding also suppresses noise within the neural signal 110, making the pulse train more robust to noise since the information is distributed over time. In such regard, the neural encoder 120 can generate a pulse train 130 to reduce the bandwidth needed to represent the neural signal 110 prior to wireless transmission. Accordingly, this reduces the amount of power needed to transmit the signal and allows the neural encoder 120 to be a small implantable medical diagnostic device.

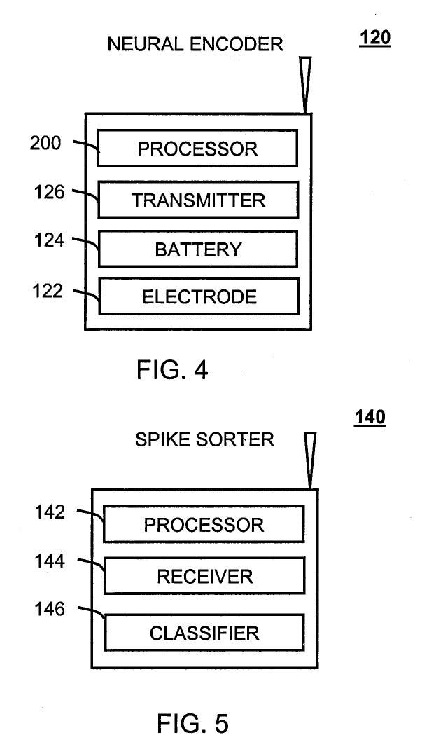

Referring now to FIG. 4, a block diagram of the neural encoder 120 is shown. The neural encoder 120 is not limited to the components shown and can include more or less than those shown. As shown, the neural encoder 120 can include an electrode 122 for acquiring neural signals, a processor 200 for compressing the neural signals to a pulse train, a transmitter 126 for sending the pulse train to a receiver located away from the neural encoder 120, and a battery for powering the neural encoder 120. The neural encoder 120 encodes information about spikes in the pulse train instead of directly transmitting the neural signal for purposes of bandwidth compression. This reduces the bandwidth required to transmit the spike trains since spike occurrences are sparse within neural signals. Moreover, the transmitting of the pulse train offers low power transmission options such as ultra wideband coding. The neural encoder 120 can adjust the timing and number of pulses based on the area of the waveform to represent the spike.

Briefly, the processor 200 encodes information concerning the spikes and a time of the spikes. The processor 200 then produces pulses in the pulse train 130 based on the spikes and the spike time. More specifically, the processor encodes features of the spikes and the time of the spikes in the timing between pulses and the number of pulses. This also suppresses the transmitting of noise information and reduces power consumption. The transmitter 126 receives the pulse train from the processor 200 and sends the pulses over a communication channel using a suitable communication protocol. For example, the transmitter 126 can include a modem (not shown) for coding the pulse train using line coding such as return-to-zero, non-return to zero, Manchester keying, bipolar return to zero keying; differential coding such as delta modulation; multilevel signaling, duo binary signaling, binary signaling including ASK, PSK, QPSK, FSK, M-ary, synchronous or asynchronous signaling and other suitable modulation schemes. Other modulations and communication techniques (e.g., Wi-Fi, Bluetooth, ZigBee, or other IEEE 802.X protocols) can be implemented for transmitting the pulse train 130.

Referring to FIG. 5, the spike sorter 140 is shown. The spike sorter 140 is not limited to the components shown and can include more or less than those shown. Moreover, the neural recording system 100 of FIG. 1 is not limited to only using the spike sorter 140 to receive the pulse train 130. The spike sorter 140 is merely shown as providing one particular embodiment for providing a portion of a neurophysiologic data acquisition system. In fact, the spike sorter 140 can be one component of a larger analysis system that receives the pulse train 130 from the neural encoder 120.

The spike sorter 140 can include a receiver 144 for receiving the pulse train 130 transmitted by the neural encoder 120, a processor 142 for analyzing and identifying spikes from the pulse train 130, and a classifier for associating the spikes with at least one neuron. Briefly, the neural encoder 120 extracts enough features from spikes in the neural signal as are needed to allow the spike sorter 140 to identify which neuron produced which spike. Notably, different neurons generate the different spikes within the neural signal 110. One assumption in spike sorting is that each neuron generates a spike signature which is characteristic of the neuron. That is, each spike can have certain features, such as an area, amplitude, or shape that are specific to the neuron generating the spike. The spike sorter 140 can categorize spikes based on their features and identify neurons associated with those features. Furthermore, the spike sorter 140 can determine which neuron fired a spike and when the neuron fired the spike. The term “fire” can be defined as generating a pulse. As an example, referring again to FIG. 3, an outcome of the spike sorter 140 can identify neuron A as having produced spike 112, and neuron B as having produced spike 113.

Briefly, returning to FIG. 1, once the pulse train 130 has been received, the spike sorter 140 can then perform spike sorting outside the acquisition zone (e.g., cortex) where power limitations are not as critical. The encoded pulses for each spike serve as a spike signature, where a spike-based spike sorting algorithm then classifies the spike. Returning back to FIG. 5, the classifier 146 can be trained once in an initial setup and periodically retrained by sending short segments of the neural signal from one electrode at a time. In one embodiment, the spike sorting algorithm can convolve the pulse train 130 with a function, such as a Gaussian function, to produce an envelope, and then compare the envelope to a template for classifying a spike signature and identifying a neuron. The term “classifying” can be broadly defined as assigning a spike to a particular class, wherein the class can be a specific neuron or type of neuron. As an example, the spike sorter 140 can distinguish between the spike signatures of two neurons encoded by the neural encoder 120 both exhibiting same spike areas—the region under the curve of a spike (e.g. integration area). The spike sorter 140 can distinguish a taller and narrower spike as having more spikes in a given time period than a shorter and wider spike. The term “integration” can be defined as a cumulative sum.

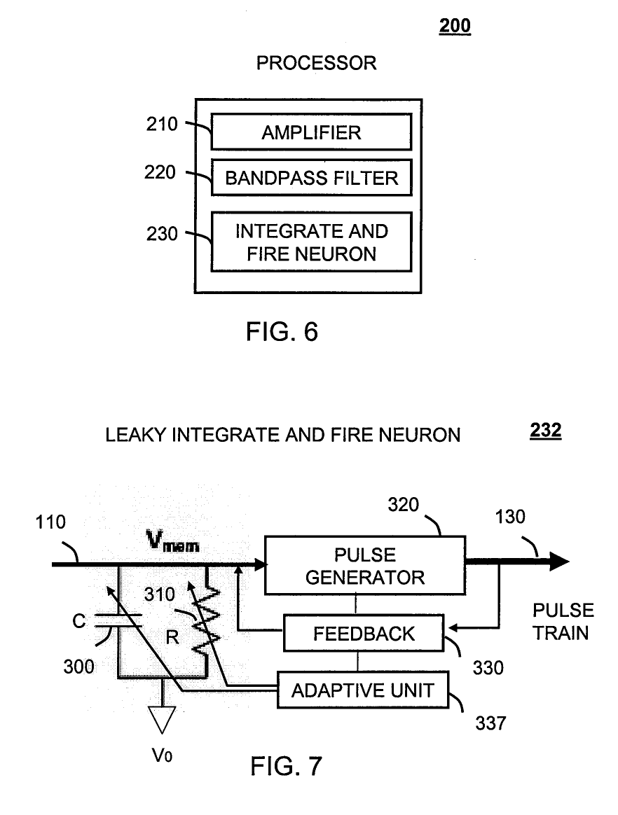

Referring to FIG. 6, a block diagram for the processor 200 of the neural encoder 120 is shown. The processor 200 is not limited to the components shown, and may include more or less than the number shown. The processor 200 can include an amplifier 210 for increasing the dynamic range of the neural signal prior, a band-pass filter 220 for filtering out noise from spikes in the neural signal, and an integrate-and-fire (IF) neuron 230 for generating a pulse train from the neural signal. The Integrate-and-Fire (IF) neuron can model waveform characteristics of a spike through timing information, wherein the timing information between the pulses captures one or more feature characteristics of the spike.

The amplifier 210 can be a voltage-to-current converter for converting a voltage signal of an electrode to a current signal, which can be separate from the processor. In practice, the amplifier 210 increases the gain of the neural signal. The amplified signal is then filtered by the band pass filter 220 to remove noise outside the frequency range of neural spikes. The IF circuit 230 then encodes the neural signal's area in a pulse train which contains spike signatures. The IF circuit 230 performs spike detection in the process of generating the pulse train.

As an example, the processor 200 can be implemented entirely in analog hardware such as a CMOS design which allows for continuous sampling, though is not limited to such. Alternatively the processor can be implemented in digital hardware or a hybrid combination of analog and digital hardware and software. For example, the processor 200 can be implemented in other digital designs such as ASIC or FPGAs, or in software on a Digital Signal Processor (DSP). The processor may include other components not shown such as an internal (on-chip read-only memory) ROM, an internal (on-chip random access memory) RAM, an internal (on-chip) flash memory, or any other memory structure.

Briefly, the IF circuit 230 receives the neural signal and encodes an integration of the waveform into a pulse train. In one arrangement, the IF circuit 230 only uses pulses of the same amplitude to communicate information about the spikes while suppressing the noise. The IF circuit 230 can significantly reduce the bandwidth of the neural signal to permit wireless transmission of the signal outside the acquisition zone of the patient (e.g., cortex area) without sacrificing the option to spike sort or increase spike detection accuracy using post processing. The IF circuit 230 also includes a leakiness aspect to increase robustness to noise and to allow synchronizing of spike signatures at the spike sorter 140 (See FIG. 5). For example, the leakiness can set an area per time threshold to filter out noise while preserving the spikes.

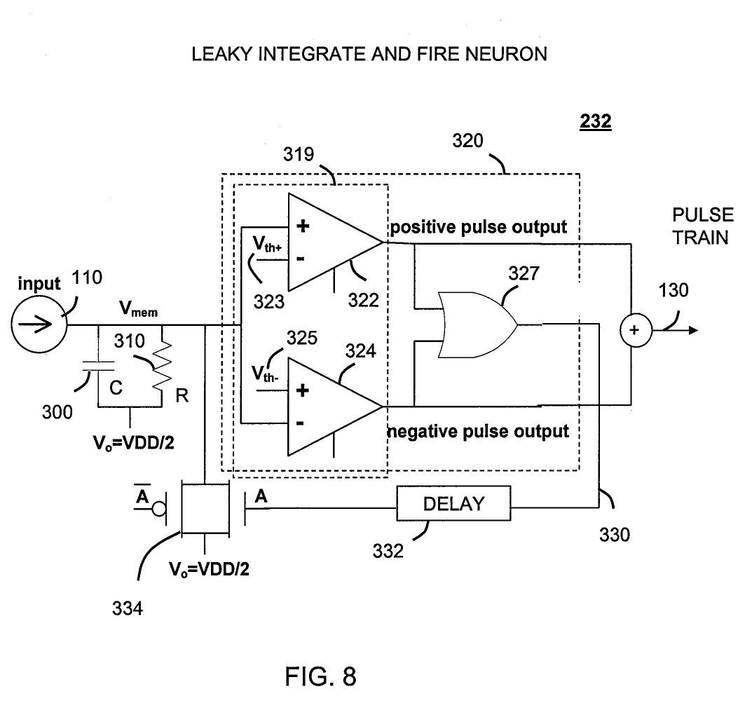

Referring to FIG. 7, a generic schematic of a Leaky Integrate and Fire (LIF) circuit 232 is shown. As shown, the LIF circuit 232 can include a capacitor 300 for integrating the neural signal 110, a resistor 310 in parallel with the capacitor 300 for providing a leakiness to the integrating of the neural signal, a pulse generator 320 for generating the pulse train 130, and a feedback unit 330 for adjusting a bandwidth compression of the neural signal. Broadly stated, the pulse generator 320 generates pulses as a function of the charge on the capacitor 300. In particular, the resistor 310, which provides a leakiness to the integration, changes the rate at which the capacitor 300 charges up. Upon charging up, the pulse generator 320 then produces a pulse. The feedback unit 330 can reset the pulse generator 320 to an initial state after generating the pulse. The term “leaky integration” can be defined as introducing a time-varying loss in the integration.

The LIF circuit 232 can also include an adaptive unit 337 for monitoring a pulse rate and adjusting the timing between pulses and the number of pulses to provide bandwidth compression. For example, the adaptive unit 337 can adjust the resistance of the resistor 310 and the capacitance of the capacitor 310 to adjust the rate and number of pulses generated by the pulse generator 320.

Referring to FIG. 8, an exemplary circuit for the LIF circuit 232 is shown in greater detail. The LIF circuit 232 includes the capacitor 300 for integrating a neural signal 110, the resistor 310 in parallel with the capacitor 300 for introducing a leakiness to the integrating, and the pulse generator 320 for producing the pulse train 130 from the integration. Recall that features of spikes within the neural signal 110 are encoded as a timing between pulses of the pulse train and a number of pulses such that the timing between pulses and the number of pulses represents features of the spike. The pulse generator 320 generates pulses in accordance with the integration. In particular, the pulse generator 320 determines the time, polarity, and the number of pulses based on the capacitor charge.

The pulse generator 320 includes a bi-phasic comparator 319 and an OR gate 327. The term “bi-phasic” can be defined as having a positive component and a negative component. The bi-phasic comparator 319 determines when the charge on the capacitor 300 exceeds a threshold. The bi-phasic comparator 319 generates a positive pulse output and a negative pulse output when the integration exceeds at least one threshold. The LIF circuit can integrate the neural signal and produces a positive pulse when the integrated signal rises above one threshold and a negative signal when it falls below a second threshold. The leakiness of the LIF circuit 232 sets an area per time threshold to filter out noise while preserving the spikes. This allows the noise in the signal to only trigger an occasional stray pulse, and thus keeps the power consumption low. The bi-phasic comparator 319 can include a first comparator 322 for generating a positive pulse output. The first comparator includes a first adjustable threshold 323 for setting a pulse rate based on a positive area of a spike. The bi-phasic comparator 319 can also include a second comparator 324 for generating a negative pulse output. The second comparator includes a second adjustable threshold 325 for setting a pulse rate based on a negative area of a spike. The OR gate 327 is coupled to the positive pulse output and negative pulse output and generates a bi-phasic output pulse train 130. Notably, the LIF neuron 232 can generate the bi-phasic output pulse train 130 asynchronously. The term “asynchronous” can be defined as without explicit dependence on a discrete or fixed clock signal or other time based referenced. The permits the neural encoder and the spike sorter to operate without explicit dependence on a discrete or fixed clock signal or other time based reference.

The bi-phasic output pulse train includes a positive pulse component from the output of the first comparator 322 and a negative pulse component from the output of the second comparator 324. The feedback 330 can also include a delay element 322 to adjust a timing between pulses of the pulse train 130 for modeling a neural refractory period. Introducing a delay in the feedback 332 delays the time at which the pass gate 334 resets the charge on the capacitor 300. Notably, the pass gate 334 resets the charge on the capacitor 300 to reset the integration. In another embodiment, the adaptive unit 337 can adjust the first threshold 323 and the second threshold 325, and the delay element 332 in the feedback unit 330 for adjusting a bandwidth compression of the neural signal 110. It should also be noted that multiple LIF circuits 232 can be combined together to increase spike detection accuracy and resolution. The neural encoder can include a bank of LIF circuits 232 that are each tuned to different frequency bands to span a range of a spike. One advantage mentioned for this multi-scale approach is that different thresholds can be set on each scale since spikes of different widths have different optimal thresholds.

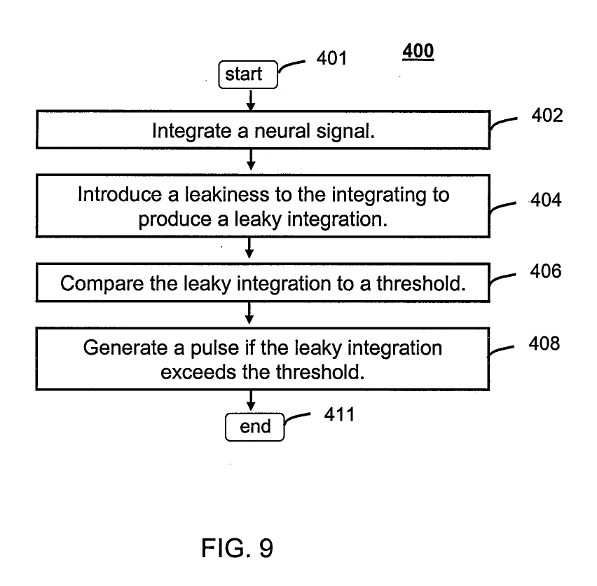

Referring to FIG. 9, a method 400 is shown for neural encoding. The method 400 can be practiced with more or less than the number of steps shown. To describe the method 400, reference will be made to FIGS. 8, 10-14, and 16 although it is understood that the method 300 can be implemented in any other suitable device or system using other suitable components. Moreover, the method 400 is not limited to the order in which the steps are listed in the method 400 In addition, the method 400 can contain a greater or a fewer number of steps than those shown in FIG. 9.

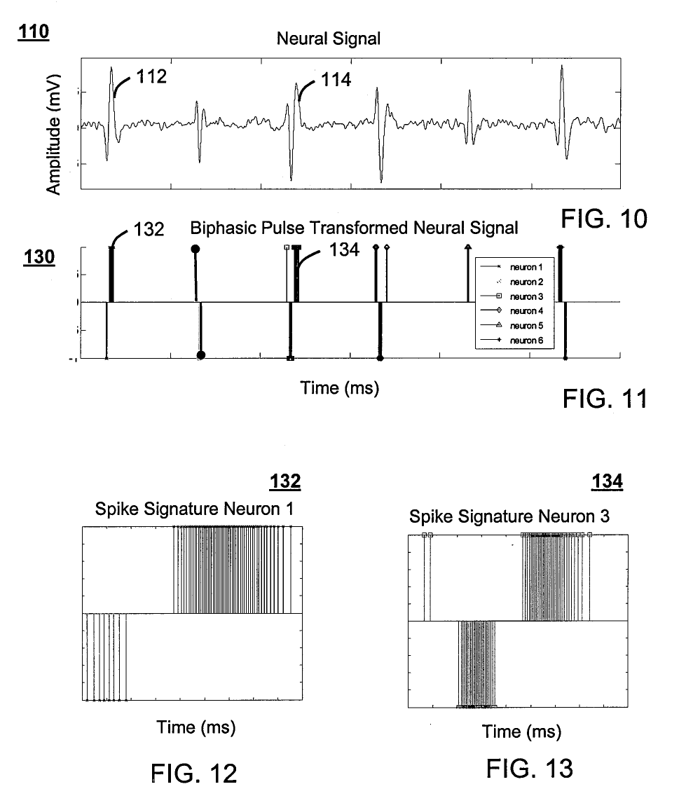

At step 401, the method 400 can begin. At step 402, a neural signal can be integrated. Referring to FIG. 10, a neural signal 110 having multiple spikes is shown. The spikes can correspond to different neurons. For example, spike 112 can correspond to a first neuron, and spike 114 can correspond to a second neuron. The spikes can be integrated using the LIF circuit 232 of FIG. 8. For example, referring to FIG. 8, the capacitor 300 charges up in accordance with a current level of the neural signal 110. The charging of the capacitor 300 corresponds to one aspect of the integrating. At step 404, a leakiness aspect can be introduced in the integration to provide a leaky integration. For example, referring to FIG. 8, the resistor 310 changes the rate at which the capacitor 300 can charge up due to charge loss. The resistor 310 provides a leakiness aspect which changes the rate and number of pulses produced by the pulse generator 320. At step 408, the leaky integration can be compared to a threshold. For example, referring to FIG. 8, the first comparator 322 can compare the capacitor charge to the first threshold 323. Similarly, the second comparator 324 can compare the capacitor charge to a second threshold 325. At step 408, a pulse can be generated if the leaky integration exceeds the voltage threshold. For example, referring to FIG. 8, the first comparator 322 can generate a positive pulse if the charge (e.g. voltage build-up of the capacitor 300) exceeds the first threshold 323. Similarly, the second comparator 322 can generate a negative pulse if the charge exceeds (in absolute terms) the second threshold 323. Positive pulses and negative pulses can be combined by the OR gate 327 to produce the bi-phasic output pulse train 130. At step 411 the method 400 can end.

Briefly, referring to FIG. 11 each spike in the neural signal 110 can be represented as a group of pulses. For instance, a first spike 112 can correspond to bi-phasic pulse sequence 132 in the pulse train 130. Briefly, referring to FIG. 12, a zoomed in view of the bi-phasic pulse sequence 132 is shown. Notably, the sequence 132 consists of a number of pulses having various timing intervals (i.e. spacing between pulses). Similarly, a second spike 114 can correspond to the bi-phasic pulse sequence 132 in the pulse train. Briefly, referring to FIG. 13, a zoomed in view of the bi-phasic pulse sequence 134 is shown.

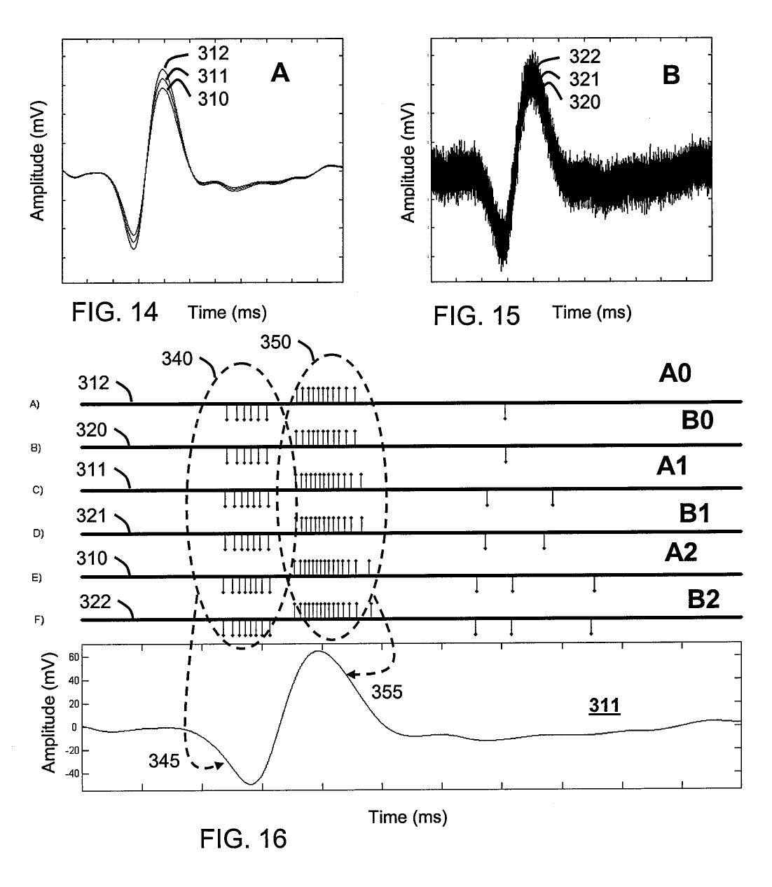

Referring to FIG. 14, multiple variations in the shape of a spike 15 are shown for demonstrating a robustness of the method 400 to temporal based pulse coding. In particular, an original spike A1 (311) having an associated width and height is slightly perturbed in one direction to produce a high-amplitude spike A0 (312) having a greater height, and in another direction to produce a low-amplitude spike A2 (310) having a lower height. Briefly, the perturbing of a spike 311 is presented to demonstrate a robustness of the method 400 for encoding a spike as a temporal-based pulse train. That is, simulation results are provided herein to demonstrate that the method 400 produces an output pulse train that is resilient to changes in the original spike 311 which can be due to noise. Referring to FIG. 15, the original spike 311, the high-amplitude spike 312 and the low-amplitude spike 310 are shown with noise. Understandably, noise can be introduced in the acquisition of neural signals which can degrade the signal quality of the recorded signal. A robustness of the encoding of the noise signals of FIG. 15 are compared to the signal variations of FIG. 14 as shown in FIG. 16.

Referring to FIG. 16, a comparison of pulse trains is presented to demonstrate the robustness of the neural encoding method 400 of FIG. 9. In particular, the location of the pulses for each paired comparison are close indicating the method 400 performed by the neural encoder 120 accurately encodes salient features of a spike. Upon the neural encoder 120 generating the pulse train 130 in accordance with the steps of method 400, the neural encoder 120 can transmit the bandwidth compressed neural signal (i.e. bi-phasic pulse train) to the spike sorter 140. The spike sorter 140 can then sort the spikes and identify neurons associated with the spikes.

For example, the first pair of encoded pulse trains shown in subplots A and B for the low-amplitude spike A0 (310) and corresponding noise spike BO (320) show similar pulse locations. Similar pulse locations indicate the neural encoder 120 is robust to amplitude and noise distortion of the neural signal. The second pair shown in subplots C and D for the original spike A1 (311) and corresponding noise spike B1 (321) show similar pulse locations. The high-amplitude pair shown in subplots E and F for the third spike A2 (312) and corresponding noise spike B2 (322) show similar pulse locations. Moreover, the dual polarity of the pulse train can be associated with the polarity of the spike in the neural signal. For example, the collection of positive output pulses 350 correspond to a positive area 355 in the original spike 311, and the collection of negative pulses 340 correspond to a negative area 345 in the original spike 311.

Salient Aspects:

1) The neural encoder 120 can process the neural signal and encode an integral of the neural signal waveform into a biphasic pulse train. One aspect of the neural encoder includes a leaky integrate-and-fire (LIF) circuit.

2) The neural encoder 120 in one arrangement only uses pulses to communicate information about the spikes while suppressing the noise. Unlike spike detection, this allows for later spike sorting.

3) The neural encoder 120 can dramatically reduce the bandwidth of the neural signal thereby allowing wireless transmission of the signal outside the patient. This preserves the option to spike sort or increase spike detection accuracy with post processing techniques.

4) The neural encoder 120 can be implemented in an ultra-low power architecture allowing for long-term implantation in the body without frequent battery replacement or elaborate through-the-skin battery recharging mechanism.

5) The neural encoder 120 encodes more information about the spike than just the height, width, and area in that it captures the attributes of spike production characteristic of the neuron producing the spike.

6) The neural encoder 120 can combine a hard thresholding of the spike detection step (e.g. the LIF circuit's leakiness) with the ability for further spike sorting, which allows some false alarms to be reclassified as noise and improve detection.

Such aspects can reduce the neural encoder's 120 susceptibility to noise which are common for long term neural recordings. Moreover, when paired with a multi-scale approach (e.g. multiple band pass versions of the original signal), the neural encoder 120 allows each scale's threshold to be set separately thereby increasing the overall performance of the system as each scale has a different optimal threshold value.

It is to be understood that the disclosed embodiments are merely exemplary, and that the invention can be embodied in various forms. Therefore, specific structural and functional details disclosed herein are not to be interpreted as limiting, but merely as a basis for the claims and as a representative basis for teaching one skilled in the art to variously employ the embodiments of the present invention in virtually any appropriately detailed structure. Further, the terms and phrases used herein are not intended to be limiting but rather to provide an understandable description of the embodiment herein. The terms “program,” “software application,” and the like as used herein, are defined as a sequence of instructions designed for execution on a computer system. A program, computer program, or software application can include a subroutine, a function, a procedure, an object method, an object implementation, an executable application, a source code, an object code, a shared library/dynamic load library and/or other sequence of instructions designed for execution on a computer system.

Where applicable, the present embodiments of the invention can be realized in hardware, software or a combination of hardware and software. Any kind of computer system or other apparatus adapted for carrying out the methods described herein are suitable. A typical combination of hardware and software can be a mobile communications device with a computer program that, when being loaded and executed, can control the mobile communications device such that it carries out the methods described herein. Portions of the present method and system can also be embedded in a computer program product, which comprises all the features enabling the implementation of the methods described herein and which when loaded in a computer system, is able to carry out these methods.

While the preferred embodiments of the invention have been illustrated and described, it will be clear that the embodiments of the invention is not so limited. Numerous modifications, changes, variations, substitutions and equivalents will occur to those skilled in the art without departing from the spirit and scope of the present embodiments of the invention as defined by the appended claims.

What is claimed is:

1. A neural acquisition system, comprising
a neural encoder that receives a neural signal and generates a temporal-based pulse-coded representation of the neural signal; and
a spike sorter communicatively coupled to the neural encoder for receiving the temporal-based pulse coded representation, sorting spikes encoded in the temporal-based pulse coded representation, and identifying neurons generating the spikes,
wherein the neural encoder encodes features of the spikes as a timing between pulses and a number of pulses such that the timing and the number of pulses represent features of the spikes that are characteristic of neural signals produced by the neurons.

2. The neural acquisition system of claim 1, wherein the neural encoder generates a temporal-based pulse coded representation of spikes in the neural signal based on integrate-and-fire coding of the received neural signal.

3. The neural acquisition system of claim 1, wherein the neural encoder further comprises:
a processor for generating the temporal-based pulse coded representation of spikes in the neural signal;
a transmitter operatively coupled to the processor for wirelessly communicating the temporal-based pulse coded representation; and
a power source for powering the processor and the wireless module.

4. The neural acquisition system of claim 1, wherein the spike sorter further comprises:
a receiver for wirelessly receiving the temporal-based pulse coded representation from the neural encoder.

5. The neural acquisition system of claim 1, wherein the neural encoder and the spike sorter operate without explicit dependence on a discrete or fixed clock signal or other time based reference.

6. The neural acquisition system of claim 1, wherein the spike sorter operates directly on the timing of pulses for sorting spikes and avoids reconstruction of the neural signal.

7. The neural acquisition system of claim 1, wherein the spike sorter further comprises:
a classifier for synchronizing spike signatures, comparing the spike signatures to templates associated with neurons, and identifying a neuron producing a spike signature.

8. The neural acquisition system of claim 7, wherein the classifier
convolves a pulse train with a Gaussian function to produce an envelope; and
compares the envelope to at least one template to identify a neuron.

9. A neural encoder suitable for use in bandwidth compression of a neural signal, the neural encoder comprising:
an Integrate and Fire (IF) circuit coupled to an electrode that captures a neural signal, wherein the IF circuit encodes at least one spike of the neural signal and generates a bi-phasic pulse train in accordance with features of the spike,
wherein the IF circuit introduces a timing between pulses of the pulse train and a number of pulses for encoding at least one feature of the waveform.

10. The neural encoder of claim 9, wherein the IF circuit includes a leaky integrator that increases a robustness to noise and allows for synchronizing spike signatures.

11. The neural encoder of claim 9, wherein the IF circuit decreases a period of the pulses for high-amplitude spikes, and increases the period of the pulses for low-amplitude spikes for bandwidth compression.

12. The neural encoder of claim 9, wherein the IF circuit includes a spike detector for identifying spikes prior to the encoding.

13. The neural encoder of claim 9, wherein the IF circuit includes an adaptive component that adjusts the timing and number of pulses for bandwidth compression.

14. The neural encoder of claim 9, wherein the IF circuit models an area of the waveform as a feature to determine the timing between pulses and the number of pulses for representing a spike.

15. The neural encoder of claim 9, wherein the IF circuit models an amplitude of the waveform as a feature to determine the timing between pulses and the number of pulses for representing a spike.

16. The neural encoder of claim 9, wherein the IF circuit includes at least one user setting for adjusting a bandwidth compression of the pulse train.

17. The neural encoder of claim 9, further comprising a bank of Integrate and Fire (IF) neuron models tuned to different frequency bands to span a range for temporal-based pulse coding of the neural signal.

18. An Integrate and Fire (IF) circuit suitable for use in bandwidth compression of a neural signal, comprising:
a leaky integrator to integrate a neural signal to produce an integrated signal;
a pulse generator to produce a bi-phasic pulse train based on the integrated signal,
wherein features of the neural signal are encoded as a timing between pulses of the pulse train and a number of pulses such that the timing between pulses and the number of pulses conveys features of the spike.

19. The IF circuit of claim 18, further comprising an amplifier operatively coupled to the capacitor and providing input to the capacitor for increasing a gain of the neural signal prior to the integrating.

20. The IF circuit of claim 18, wherein the leaky integrator comprises:
a capacitor for building up a charge in accordance with a voltage of the neural signal; and
a resistor coupled in parallel with the capacitor that leaks of a portion of the charge, wherein the resistor provides a leakiness to the integrating by decreasing the charge on the capacitor over time.

21. The IF circuit of claim 18, wherein the pulse generator further comprises:
a bi-phasic comparator for generating a positive pulse output when the integrated signal exceeds a first threshold, and a negative pulse output when the integrated signal exceeds a second threshold; and
an OR gate coupled to the positive pulse output and negative pulse output for generating a bi-phasic output pulse train,

22. The IF circuit of claim 18, wherein the bi-phasic comparator further includes
a first comparator for generating a positive pulse output, wherein the first comparator includes a first adjustable threshold for setting a pulse rate based on a positive area of a spike; and
a second comparator for generating a negative pulse output, wherein the second comparator includes a second adjustable threshold for setting a pulse rate based on a negative area of a spike;

23. The IF circuit of claim 18, further comprising:
a feedback unit coupling the output of the amplifier to the input of the amplifier for resetting the pulse generator to an initial state

24. The IF circuit of claim 23, wherein the feedback unit includes a delay element to adjust a timing between pulses of the pulse train for modeling a neural refractory period.

25. The IF circuit of claim 23, wherein the feedback unit includes an adaptive unit for monitoring a pulse rate and adjusting the timing between pulses and the number of pulses for bandwidth compression.

26. The IF circuit of claim 26, wherein the adaptive unit adjusts at least one of a resistance of the resistor, a capacitance of the capacitor, a threshold of the bi-phasic comparator, or a delay of a feedback for bandwidth compression of the pulse train.

27. A method for neural encoding, comprising:
integrating a neural signal to produce an integrated signal;
generating a pulse if a level of the integrated signal exceeds a threshold,
wherein characteristic features of the spikes are encoded as a timing between pulses and a number of pulses such that the timing and number of pulses represents features of the neural signal.

28. The method of claim 27, further comprising:
detecting a spike in the neural signal prior and encoding features of the spike to increase bandwidth compression of the neural signal.

29. The method of claim 27, further comprising:
introducing a leakiness to the integrating to produce a leaky integration.

30. The method of claim 27, further comprising:
wirelessly transmitting the pulse train asynchronously to a spike sorter, wherein the pulse train provides bandwidth compression of the spike.

31. The method of claim 27, further comprising:
enabling a power amplifier to transmit a pulse upon the integrating exceeding a threshold; and
keeping the power amplifier in power save mode otherwise.

32. The method of claim 27, further comprising:
sorting spikes encoded within the timing of the pulse train without reconstructing the neural signal.

33. The method of claim 27, wherein the generating further comprises:
comparing the integration to a positive threshold and generating a positive pulse if the integration exceeds the positive threshold; and
comparing the integration to a negative threshold and generating a negative pulse if the integration exceeds the negative threshold,

34. The method of claim 27, wherein the generating a pulse train further comprises:
adjusting a pulse rate and number of pulses in accordance with an area of a waveform of the spike.

35. The method of claim 27, wherein the generating a pulse train further comprises:
adjusting a pulse rate and number of pulses in accordance with an amplitude of a waveform of the spike.

36. The method of claim 27, wherein the generating a pulse train further comprises:
introducing a delay in a feedback of the pulse train for modeling a refractory period.

37. The method of claim 27, wherein the generating a pulse train further comprises:
adapting a threshold in accordance with the timing between pulses and the number of pulses for modeling inhibition and excitation.

38. The method of claim 27, wherein the generating a pulse train suppresses noise on the spike.
